SEQUENCE LISTING (6 sequences)

SEQ ID NO: 1 - DNA, 2071 bp, Artificial Sequence
  Synthetic
actgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgatgagggcccaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtcaagcagctgcaggtgtgtgtgtgtgaggctcgtcctgaatgatatcaagcttgaattcgttgacgaattctctagatatcgctcaatcacacacacacctgcagctcatc

SEQ ID NO: 2 - DNA, 146 bp, Artificial Sequence
  Synthetic
atccggcaaacaaaccaccgctggtagcggtggtttttttgtttgcaagcagcagattacgcgcagaaaaaaaggatctcaagaagatcctttgattttctaccgaagaaaggcccacccgtgaaggtgagccagtgagttgattg

SEQ ID NO: 3 - DNA, 41 bp, Artificial Sequence
  Synthetic
gcaaacaaaccaccgctggtagcggtggtttttttgtttgc

SEQ ID NO: 4 - DNA, 44 bp, Artificial Sequence
  Synthetic
cgcagaaaaaaaggatctcaagaagatcctttgattttctaccg

SEQ ID NO: 5 - DNA, 158 bp, Artificial Sequence
  Synthetic
tccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattca

SEQ ID NO: 6 - DNA, 34 bp, Artificial Sequence
  Synthetic
aagtcaaaagcctccgaccggaggcttttgactt

(19) United States
(12) Patent Application Publication
(10) Pub. No.: US 2018/0355353 A1
(43) Pub. Date: Dec. 13, 2018

(54) TRANSCRIPTION TERMINATOR AND USE THEREOF

(71) Applicant: Gen9, Inc., Boston, MA (US)

(72) Inventor: Ishtiaq E. Saaem, Chelsea, MA (US)

(73) Assignee: Gen9, Inc., Boston, MA (US)

(21) Appl. No.: 15/779,902

(22) PCT Filed: Nov. 29, 2016
(86) PCT No.: PCT/US2016/063955

§ 371 Date: May 30, 2018

Related U.S. Application Data
(60) Provisional application No. 62/260,700, filed on Nov. 30, 2015.

(51) Int. Cl. C12N 15/113; C12N 15/63

(52) U.S. Cl. CPC C12N 15/113; C12N 15/63

(57) ABSTRACT

Artificial transcription terminators and their use are provided herein. In one aspect, a non-naturally occurring nucleic acid sequence can comprise a Y-X-Z stem-loop, wherein: Y is a nucleotide sequence of 10 to 30 nucleotides in length; X is a nucleotide sequence of 3 to 12 nucleotides in length, each nucleotide therein not base pairing with any other nucleotide within X; and Z is a nucleotide sequence of 10 to 50 nucleotides in length and having at least 70% complementarity to Y.

### CROSS-REFERENCE TO RELATED APPLICATIONS

This application claims the benefit of and priority to U.S. Provisional Patent Application No. 62/260,700 file Nov. 30, 2015, the entire disclosure of which is incorporated herein by reference.

### FIELD

The present disclosure relates in general to non-naturally occurring, synthetic genetic components useful for molecular cloning. More particularly, artificial transcription terminators are provided, for use in cloning natural or non-natural DNA inserts.

### BACKGROUND

In molecular cloning and recombinant DNA technology, various DNA inserts such as a gene or fragment thereof are often introduced into a vector which is then multiplied in a host culture. However, undesirable expression of the DNA inserts, e.g., into secondary proteins unnecessary for survival, is detrimental to host health and growth. A controllable expression system is desired that allows the specific adjustment of expression rate and of modifications to the cell metabolism. One important aspect of recombinant expression systems is transcriptional efficiency, including efficiency of termination. Low termination efficiency leads to read-through transcription and the production of lengthy mRNAs that by themselves are stressful to a cell, but even more so can lead to the expression of unwanted proteins or disturb the replication control of the transgene construct—in particular in the field of plasmid vectors.

Thus, a need exists for improved vector components such as transcription terminators, in particular synthetic, non-naturally occurring terminators that have high termination efficiency.

### SUMMARY

The present disclosure provides vectors and vector components configured for multiplex cloning, multiplex sequencing, and fixed orientation cloning. The vector and vector components described herein allow insert sequences that can be deleterious to a host to be successfully cloned. The vector described herein also combats the disadvantage of direct selection vectors that contain a promoter that actively transcribes the region into which the insert DNA is to be cloned. In some embodiments, a low-background vector that does not transcribe the inserted DNA fragment is provided. Therefore, insert DNA that encodes toxic or otherwise deleterious peptides or proteins that are harmful or stressful to the host in which it is carried can be tolerated by the host.

In one aspect, one or more non-naturally occurring, artificial transcription terminator can be included in a vector, either as part of the vector to which the insert is introduced, or as part of the insert that is synthesized or assembled. The transcription terminators provided herein can be used to facilitate the cessation of transcription of a transcript (e.g., an mRNA transcript). In some embodiments, the transcription terminator can include one or more stem-loop sequence.

In some embodiments, the present disclosure provides a non-naturally occurring nucleic acid sequence comprising a Y-X-Z stem-loop, wherein: Y is a nucleotide sequence of 10 to 30 nucleotides in length; X is a nucleotide sequence of 3 to 12 nucleotides in length, each nucleotide therein not base pairing with any other nucleotide within X; and Z is a nucleotide sequence of 10 to 50 nucleotides in length and having at least 70% complementarity to Y.

In some embodiments, Y has a G/C content of at most 60%, at most 50%, or at most 40%. Y may be 5′ to X or 3′ to X. Y can be, in certain embodiments, 12-18 nucleotides in length, 14-16 nucleotides in length, 16-18 nucleotides in length, 17-19 nucleotides in length, 15-30 nucleotides in length, 18-27 nucleotides in length, 21-24 nucleotides in length, 24-28 nucleotides in length, or 25-29 nucleotides in length.

X is the loop portion of the stem-loop and may be 3-8 nucleotides in length, 4-6 nucleotides in length or 5-6 nucleotides in length in some embodiments.

Z can have the same or different length as Y. Z may have one or more mismatches with Y. Z can also have one or more insertions or deletions compared to Y, thereby forming a protrusion or loop when annealed with Y.

The stem-loop in some embodiments can include the sequence of AAGC and/or CATC. In some examples, the stem-loop can have the sequence of SEQ ID NO: 3, 4, or 6.

A further aspect relates to a transcription terminator comprising a first stem-loop and a second stem-loop, wherein the first stem-loop has any one of the non-naturally occurring stem-loop nucleic acid sequences disclosed herein, and wherein the first stem-loop is 5′ to the second stem-loop. In some embodiments, the second stem-loop is a short stem-loop. The second stem-loop may also have any one of the non-naturally occurring stem-loop nucleic acid sequences disclosed herein. The transcription terminator can further include a third stem-loop which can be a short stem-loop or have any one of the non-naturally occurring stem-loop nucleic acid sequence disclosed herein. In some embodiments, the transcription terminator have the sequence of SEQ ID NO: 2 or 5.

Also provided herein is a vector comprising one or more transcription terminators disclosed herein, operably linked to a DNA insert. The vector in one embodiment has the sequence of SEQ ID NO: 1. The DNA insert can be any nucleic acid of interest (e.g., for cloning purpose) such as a gene, a gene fragment, and an open reading frame. In some embodiments, the DNA insert is a non-naturally occurring nucleic acid molecule. In certain embodiments, any portion of the vector such as the DNA insert and/or transcription terminator can be a synthetic molecule made by, e.g., various synthesis and assembly strategies as described in, for example, PCT Publication Nos. WO2014/151696, WO2014/004393, WO2013/163263, WO2013/032850, WO2012/078312, WO2004/24886, WO2008/027558, WO2010/025310, and WO2016/064856, the disclosures of all of which are hereby incorporated by reference in their entirety.

Another aspect relates to an engineered cell comprising the vector disclosed herein.

A further aspect related to a method of engineering a vector, comprising providing any transcription terminator disclosed herein in a vector, wherein the transcription terminator is engineered to operably link to a DNA insert.

A further aspect relates to a method of terminating transcription of a DNA insert, comprising: (a) providing any transcription terminator disclosed herein engineered to operably link to the DNA insert; (b) allow transcription of the DNA insert; and (c) terminate transcription of the DNA insert at the transcription terminator.

### BRIEF DESCRIPTION OF THE DRAWINGS

FIG. 1 illustrates an exemplary vector map.

FIG. 2 illustrates a schematic of a vector having two terminators (“T”).

FIG. 3 illustrates an exemplary embodiment of a transcription terminator.

FIG. 4 illustrates an exemplary embodiment of a transcription terminator.

### DETAILED DESCRIPTION

The present disclosure provides vectors, vector components and polynucleotides configured for multiplex cloning, multiplex sequencing, and/or fixed orientation cloning. In some embodiments, insert sequences that may be deleterious to a host can be successfully cloned using the polynucleotides provided herein. This is particularly advantageous during genetic engineering which often differs from natural genetics in two ways. First, very strong promoters are frequently required for synthetic circuits, generating a high flux of RNA polymerase (RNAP). Second, designs are modularly organized along a relatively short stretch of linear DNA, so to not interfere with the next transcription unit the high flux of RNAP needs to be sharply stopped. This hard start-hard stop design introduces a need for strong terminators.

In some embodiments, a low-background vector that does not transcribe the inserted DNA fragment is provided. The vector can include one or more synthetic, non-natural polynucleotide sequences having the characteristics described herein. The polynucleotide can be DNA (usually encoding the terminator) or RNA (which usually is able to fold into the hairpin structure and may comprise the terminator). The polynucleotide can be single stranded (especially for RNA) or double stranded (especially for DNA).

In certain embodiments, the polynucleotide sequence is a transcription terminator. One or more terminators can be included at 5′ and/or 3′ of the insert sequence, and/or within the insert sequence. The terminator can be built into the vector. In some embodiments, the terminator can be synthesized or assembled as part of the insert sequence which is then introduced into the vector. Various synthesis and assembly strategies are described in, for example, PCT Publication Nos. WO2014/151696, WO2014/004393, WO2013/163263, WO2013/032850, WO2012/078312, WO2004/24886, WO2008/027558, WO2010/025310, and WO2016/064856, the disclosures of all of which are hereby incorporated by reference in their entirety.

In some embodiments, following synthesis or assembly of one or more target nucleic acids, they can be individually cloned into a vector, or such cloning can be performed in a multiplex fashion in parallel. Incorporating one or more transcription terminators disclosed herein can increase cloning success rate and efficiency.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, the term “amino acid sequence” refers to a sequence of contiguous amino acid residues of any length. The terms “polypeptide,” “peptide,” “oligopeptide,” or “protein” may be used interchangeably herein with the term “amino acid sequence.”

“Copy number” of a genetic element, plasmid or vector refers to how many copies are present in a host cell. Copy number is generally determined by the origin of replication (“ORI”) used and can be manipulated with mutations in the ORI. For example, the pMB 1 ORI maintains about 20 copies per cell, while pUC—which contains a derivative of the pMB1 ORI differs by only two mutations—will produce as many as 700 copies per cell. A “high copy number” genetic element or plasmid is one that is capable of replicating itself till at least, for example, 100 copies are present per cell. Commonly used high copy number plasmids include pUC (pMB1 derivative ORI), pBluescript (ColE1 derivative ORI), and pGEM (pMB1 derivative ORI). A “low copy number” genetic element or plasmid is present at, e.g., less than about 20 copies per cell. Commonly used low copy number plasmids include pBR322 (pMB1 ORI), pET (pMB1 ORI), pGEX (pMB1 ORI), pColE1 (ColE1 ORI), pR6K (R6K ORI), pACYC (p15A ORI), pSC101 (pSC101 ORI) and pLys (p15A ORI).

A “genetic element” may be any coding or non-coding nucleic acid sequence that is capable of self replicating. Genetic elements may include one or more origins for replication, operons, genes, gene fragments, exons, introns, markers, regulatory sequences, promoters, operators, catabolite activator protein (also known as cyclic AMP receptor protein, “CAP”) binding sites, enhancers, transcriptional terminators, or any combination thereof, which can be operably linked together. Examples include plasmid, phage vector, phagemid, transposon, cosmid, chromosome, artificial chromosome, episome, virus, virion, etc. In some instances, “genetic element” and “vector” are used interchangeably.

A “host” is intended to include any individual virus or cell or culture thereof that can be or has been a recipient for vectors or for the incorporation of exogenous nucleic acid molecules, polynucleotides, and/or proteins. It also is intended to include progeny of a single virus or cell. The progeny may not necessarily be completely identical (in morphology or in genomic or total DNA complement) to the original parent cell due to natural, accidental, or deliberate mutation. The virus can be phage. The cells may be prokaryotic or eukaryotic, and include but are not limited to bacterial cells, yeast cells, insect cells, animal cells, and mammalian cells, e.g., murine, rat, simian, or human cells.

As used herein, “identity” means the percentage of identical nucleotides at corresponding positions in two or more sequences when the sequences are aligned to maximize sequence matching, i.e., taking into account gaps and insertions. Methods to determine identity are designed to give the largest match between the sequences tested. Moreover, methods to determine identity are codified in publicly available computer programs. Computer program methods to determine identity between two sequences include, but are not limited to, the GCG program package, BLASTP, BLASTN, and FASTA. The BLAST program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm may also be used to determine identity. BLASTN can e.g. be run using default parameters with an open gap penalty of 11.0 and an extended gap penalty of 1.0 and utilizing the blosum-62 matrix.

As used herein, “including,” “comprising,” “having,” “containing,” “involving,” and variations thereof herein, are meant to encompass the items listed thereafter and equivalents thereof as well as additional items. “Consisting of” shall be understood as a close-ended relating to a limited range of elements or features. “Consisting essentially of” limits the scope to the specified elements or steps but does not exclude those that do not materially affect the basic and novel characteristics of the claimed invention.

An “insert” as used herein, is a heterologous nucleic acid sequence that is ligated into a compatible site into a vector. An insert may comprise one or more nucleic acid sequences (e.g., a gene or a fragment thereof) that encode a polypeptide or polypeptides. An insert may comprise regulatory regions or other nucleic acid elements that allow, for example, transcription and/or translation of the insert.

“Nucleic acid,” “nucleic acid sequence,” “oligonucleotide,” “polynucleotide,” “gene” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc.

As used herein, an oligonucleotide may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 300 nucleotides long (e.g., from about 30 to 250, from about 40 to 220 nucleotides long, from about 50 to 200 nucleotides long, from about 60 to 180 nucleotides long, or from about 65 or about 150 nucleotides long), between about 100 and about 200 nucleotides long, between about 200 and about 300 nucleotides long, between about 300 and about 400 nucleotides long, or between about 400 and about 500 nucleotides long. However, shorter or longer oligonucleotides may be used. An oligonucleotide may be a single-stranded or double-stranded nucleic acid. As used herein the terms “nucleic acid”, “polynucleotide”, “oligonucleotide” are used interchangeably and refer to naturally-occurring or non-naturally occurring, synthetic polymeric forms of nucleotides. In general, the term “nucleic acid” includes both “polynucleotide” and “oligonucleotide” where “polynucleotide” may refer to longer nucleic acid (e.g., more than 1,000 bases or base pairs, more than 5,000 bases or base pairs, more than 10,000 bases or base pairs, etc.) and “oligonucleotide” may refer to shorter nucleic acid (e.g., 10-500 bases or base pairs, 20-400 bases or base pairs, 40-200 bases or base pairs, 50-100 bases or base pairs, etc.). The nucleic acid molecules of the present disclosure may be formed from naturally occurring nucleotides, for example forming deoxyribonucleic acid (DNA) or ribonucleic acid (RNA) molecules. Alternatively, naturally-occurring nucleic acids may include structural modifications to alter their properties, such as in peptide nucleic acids (PNA) or in locked nucleic acids (LNA). The solid phase synthesis of nucleic acid molecules with naturally occurring or artificial bases is well known in the art. The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. In some embodiments, the sequence of the nucleic acids does not exist in nature (e.g., a cDNA or complementary DNA sequence, or an artificially designed sequence).

Usually in a nucleic acid nucleosides are linked by phosphodiester bonds. Whenever a nucleic acid is represented by a sequence of letters, it will be understood that the nucleosides are in the 5′ to 3′ order from left to right. In accordance to the IUPAC notation, “A” denotes deoxyadenosine, “C” denotes deoxycytidine, “G” denotes deoxyguanosine, “T” denotes deoxythymidine, “U” denotes the ribonucleoside, uridine. In addition, there are also letters which are used when more than one kind of nucleotide could occur at that position: “W” (i.e. weak bonds) represents A or T, “S” (strong bonds) represents G or C, “M” (for amino) represents A or C, “K” (for keto) represents G or T, “R” (for purine) represents A or G, “Y” (for pyrimidine) represents C or T, “B” represents C, G or T, “D” represents A, G or T, “H” represents A, C or T, “V” represents A, C, or G and “N” represents any base A, C, G or T (U). It is understood that nucleic acid sequences are not limited to the four natural deoxynucleotides but can also comprise ribonucleoside and non-natural nucleotides. A “/” in a nucleotide sequence or nucleotides given in brackets refer to alternative nucleotides, such as alternative U in a RNA sequence instead of T in a DNA sequence. Thus, U/T or U(T) indicate one nucleotide position that can either be U or T. Likewise, A/T refers to nucleotides A or T; G/C refers to nucleotides G or C. Due to the functional identity between U and T any reference to U or T herein shall also be seen as a disclosure as the other one of T or U. For example, the reference to the sequence UUCG (on an RNA) shall also be understood as a disclosure of the sequence TTCG (on a corresponding DNA). For simplicity only, only one of these options is described herein. Complementary nucleotides or bases are those capable of base pairing such as A and T (or U); G and C; G and U.

As used herein, the terms “operably linked” or “operably positioned” means a genetic component having a first activity (e.g., terminator activity) is engineered to be in the same nucleic acid molecule, and is in a functional relationship, with another genetic component having a second activity (e.g., promoter, operator, catabolite activator protein binding site, enhancer, gene, gene fragment, open reading frame, etc.). For example, a terminator is operably linked to an insert means that the terminator and insert (e.g., a gene) are engineered together (e.g., in an expression cassette) such that transcription from the insert can be terminated at the terminator.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

“Plasmid” is a small circular piece of DNA that replicates independently from the hosts chromosomal DNA. The host can be bacteria, yeast, plant, or mammalian cells. Plasmids typically have an origin of replication, a selection marker, and one or more cloning sites. A plasmid can contain two or more different origins of replication, such that it can shuttle between two or more different hosts.

As used herein, the term “promoter” refers to a DNA sequence capable of controlling the transcription of a nucleotide sequence of interest into mRNA, and generally contains a RNA polymerase binding site and one or more operators and/or catabolite activator protein (also known as cyclic AMP receptor protein, “CAP”) binding sites for biding of other transcriptional factors. A promoter may be constitutively active (“constitutive promoter”) or be controlled by other factors such as a chemical, heat or light. The activity of an “inducible promoter” is induced by the presence or absence or biotic or abiotic factors. Commonly used constitutive promoters include CMV, EF1a, SV40, PGK1, Ubc, human beta actin, CAG, Ac5, Polyhedrin, TEF1, GDS, ADH1 (repressed by ethanol), CaMV35S, Ubi, H1, U6, T7 (requires T7 RNA polymerase), and SP6 (requires SP6 RNA polymerase). Common inducible promoters include TRE (inducible by Tetracycline or its derivatives; repressible by TetR repressor), GAL1 & GAL10 (inducible with galactose; repressible with glucose), lac (constitutive in the absence of lac repressor (LacI); can be induced by IPTG or lactose), T7lac (hybrid of T7 and lac; requires T7 RNA polymerase which is also controlled by lac operator; can be induced by IPTG or lactose), araBAD (inducible by arabinose which binds repressor AraC to switch it to activate transcription; repressed catabolite repression in the presence of glucose via the CAP binding site or by competitive binding of the anti-inducer fucose), trp (repressible by tryptophan upon binding with TrpR repressor), tac (hybrid of lac and trp; regulated like the lac promoter; e.g., tacI and tacII), and pL (temperature regulated). The promoter can be prokaryotic or eukaryotic promoter, depending on the host. Common promoters and their sequences are well known in the art.

In general, a “stem-loop” sequence (used interchangeably with “hairpin”) refers to a sequence in which at least two regions within a single nucleic acid (DNA or RNA or otherwise) molecule that are reverse compliments of each other are separated by one or more non-complimentary region, such that the complementary regions hybridize and form a “stem,” while the non-complementary region forms a “loop.”

“Termination” as used herein shall refer to transcription termination if not otherwise noted. “Termination signal” or simply “terminator” refers to a nucleic acid sequence that hinders or stops transcription of a RNA polymerase. In some embodiments, the terminators disclosed herein are used in connection with the T7 RNA polymerase but can also effect termination for other RNA polymerases.

As used herein, unless otherwise stated, the term “transcription” refers to the synthesis of RNA from a DNA template; the term “translation” refers to the synthesis of a polypeptide from an mRNA template. Transcription and translation collectively are known as “expression.”

The term “transfect” or “transform” or “transduce” as used herein refers to a process by which exogenous nucleic acid is transferred or introduced into the host cell. A transfected or transformed cell includes the primary subject cell and its progeny. The host cell can be bacteria, yeasts, mammalian cells, and plant cells.

As used herein, the term “vector” refers to a nucleic acid molecule capable of transporting another nucleic acid to which it has been linked. A vector includes any genetic element, such as a plasmid, phage vector, phagemid, transposon, cosmid, chromosome, artificial chromosome, episome, virus, virion, etc., capable of replication (e.g., containing an origin of replication which is DNA sequence allowing initiation of replication by recruiting replication machinery proteins) when associated with the proper control elements and which can transfer gene sequences into or between hosts. One type of vector is an episome, i.e., a nucleic acid capable of extra-chromosomal replication. Another type of vector is an integrative vector that is designed to recombine with the genetic material of a host cell. Vectors may be both autonomously replicating and integrative, and the properties of a vector may differ depending on the cellular context (i.e., a vector may be autonomously replicating in one host cell type and purely integrative in another host cell type). Vectors generally contain one or a small number of restriction endonuclease recognition sites and/or sites for site-specific recombination. A foreign DNA fragment may be cleaved and ligated into the vector at these sites. The vector may contain a marker suitable for use in the identification of transformed or transfected cells. For example, markers may provide antibiotic resistant, fluorescent, enzymatic, as well as other traits. As a second example, markers may complement auxotrophic deficiencies or supply critical nutrients not in the culture media.

Other terms used in the fields of recombinant nucleic acid technology, microbiology, genetic engineering, and molecular and cell biology as used herein will be generally understood by one of ordinary skill in the applicable arts.

### Transcription Terminator

Transcription is a central step of gene expression, and thus may present a powerful option to manipulate the expression of a single gene or group of genes. Transcription takes place on DNA template where an mRNA-DNA-RNA polymerase ternary structure is formed on which the RNA polymerase (RNAP) catalyzes the synthesis of mRNA transcripts. Once the ternary complex is build up, it needs be stable enough to allow the incorporation of up to hundred bases per second without dissociation of the RNAP during non-terminating transcriptional pauses or delays. Thus a tight connection of the elongating RNAP with the template DNA and the resulting RNA transcript is essential for the ability to produce mRNAs with a length of several hundred or thousand nucleotides.

After transcriptional initiation and the building up of an extraordinary stable ternary complex the RNAP enzyme moves along the template, incorporates nucleotides one by one and produces the desired mRNA chain. The synthesis of mRNA and the release of the mRNA of a single gene or transcriptional operon have to be stopped at distinct sites on the template. This process is called transcriptional termination and resembles the events during transcriptional initiation but in reversed order, resulting in the dissociation of RNAP and the release of transcribed RNA. Termination occurs in response to well-defined signals within the template DNA, the so-called transcription terminators or transcriptional terminators or simply, terminators. Like most biological processes, termination is not a make-or-break decision, and thus, does not happen in an extent of 100%. Indeed terminators vary widely in their efficiencies of termination, with great differences in termination efficiency (TE). Indeed, termination signals are highly specific for a given RNA polymerase. A non-terminating event is also described as read through of the polymerase.

Intrinsic transcription terminators or Rho-independent terminators require the formation of a self-annealing hairpin structure on the elongating transcript, which results in the disruption of the mRNA-DNA-RNA polymerase ternary complex. The natural terminator sequence contains a 20 base pair GC-rich region of dyad symmetry followed by a short poly-T tract or “T stretch” which is transcribed to RNA to form the terminating hairpin and a 7-9 nucleotide “U track” respectively. (Dyad symmetry refers generally to two areas of a DNA strand whose base pair sequences are inverted repeats of each other. They are often described as palindromes.) A survey of natural and synthetic terminators is provided in Chen et al., Characterization of 582 natural and synthetic terminators and quantification of their design constraints, Nature Methods 10, 659-664 (2013), incorporated herein by reference.

The mechanism of termination is hypothesized to occur through a combination of direct promotion of dissociation through allosteric effects of hairpin binding interactions with the RNAP and “competitive kinetics”. The hairpin formation causes RNAP stalling and destabilization, leading to a greater likelihood that dissociation of the complex will occur at that location due to an increased time spent paused at that site and reduced stability of the complex.

For a long time the stability of the hairpin mediated by G-C pairs within the stem structure was believed to be the most essential compartment of the hairpin structure to affect TE. Insertion of putative bases into the stem structure should theoretically result in a higher overall AG value, and therefore the overall TE should increase. Surprisingly the increase of thermodynamic stability by inserting G-C pairs did not result in higher TE, indicating that the stability of the hairpin structure is not the only essential determinant of termination. It is assumed that in addition to stability the three dimensional structure of the hairpin plays an important role in termination. For the most characterized intrinsic terminators the distance from the first closing base pair of the stem structure to the first termination position is conserved. That invariance could be seen as putative evidence for the importance of the three dimensional structure. As a conclusion it seems that the hairpin has to assume a distinct three dimensional shape, in order to interact with the elongating polymerase.

In one aspect, non-naturally occurring, artificial transcription terminators are provided herein. In some embodiments, the transcription terminator can include one or more stem-loop sequences. In some cases, a stem-loop sequence can be about 7 to about 200 nucleotides in length, between 10 and 100 nucleotides in length, between 15 and 80 nucleotides in length, between 20 and 50 nucleotides in length, or between 30 and 40 nucleotides in length. The stem-loop sequence may he shorter or longer depending on the design.

Within each stem-loop, one or more loop structures can be designed. The loop can be a full loop where the two nucleotides at the base of the loop and connecting with the stem are complementary (e.g., A-T or G-C). Generally the loop at the top of the stem is a full loop. The loop can also be a half loop if the two nucleotides at the base of the loop and connecting with the stem do not form a base pair (e.g., A and A, T and T, A and G, T and C, etc.). A stem-loop can have one or more full loops and/or half loops. The size of the loop, excluding the two nucleotides at the base of the loop and connecting with the stem, can be anywhere between 3-12 nucleotides, or between 4-10 nucleotides, or between 5-8 nucleotides, if the host is bacterium such as E. coli. If the host is yeast or a mammalian cell, the loop size can be larger, e.g., up to 15 nucleotides or up to 20 nucleotides or larger.

The stem portion does not need to have 100% complementarity between the two base-paring fragments. For convenience, one fragment in the stem is name positive or+fragment while the other negative or−fragment. In some embodiments, the stem can have at least about 98%, at least about 95%, at least about 90%, at least about 85%, at least about 80%, at least about 75%, at least about 70%, at least about 60%, or at least about 50% of complementarity between the two base-paring fragments. Where there is less than 100% complementarity, the positive fragment may contain, compared to the negative fragment, one or more mismatches, one or more insertions (consecutively so as to form a loop or non-consecutively) and/or one or more deletions (consecutively so as to form a loop on the negative fragment or non-consecutively).

In certain embodiments, a stem-loop sequence can be a “tall” stern-loop having a long stem or a “short” stem-loop having a short stem. In general, a tall stem-loop can have a stem that is, when folded on one strand, at least two times (2×) the size of an RNA polymerase (RNAP), e.g., 2×RNAP, 3×RNAP, or longer, or any size in between. A short stem-loop generally has a stem that is shorter than two times the size of an RNAP, e.g., 1×RNAP, 2×RNAP, or shorter, or any size in between. An RNAP can occupy about 5-10 nucleotides in length, or about 6-9 nucleotides in length, or about 7-8 nucleotides in length, which can be the length of a 1×RNAP stem. Thus, a 2×RNAP stem may be about 10-20 nucleotides in length, about 12-18 nucleotides in length, about 14-16 nucleotides in length, about 16-18 nucleotides in length, or about 17-19 nucleotides in length. A 3×RNAP stern may be about 15-30 nucleotides in length, about 18-27 nucleotides in length, about 21-24 nucleotides in length, about 24-28 nucleotides in length, or about 25-29 nucleotides in length. So on and so forth.

It should be appreciated that in some embodiments, it may be desirable to keep the terminator sequence as short as possible (while having sufficient termination efficiency) to minimize the overall size of the vector so as to accommodate large inserts. In these cases the tall stem-loop can be designed to have a stern length of no more than 3×RNAP or no more than 2×RNAP. In cases where vector size is of less concern, longer stems (e.g., 3×RNAP or longer) can be included.

A transcription terminator can include more than one stem-loop sequences. In some embodiments, a transcription terminator can have at least 2 stem-loops, at least 3 stem-loops, at least 4 stem-loops, at least 5 stem-loops, at least 6 stem-loops, or more or less. Where the host is bacterium such as E. coli, the terminator may include 3 stem-loops or less to keep the vector size small.

A transcription terminator can include a mixture of one or more tall stem-loops and one or more short stem-loops. The stem-loops within each terminator can be any combination or arrangement of tall and short stem-loops. For example, the terminator can include, from 5′ to 3′, a tall stem-loop followed by a short stem-loop and then a tall stem-loop. The terminator can also include 3 tall stem-loops. In another example, the terminator may have 6 stem-loops, in the order of tall-tall-short-short-tall-tall from 5′ to 3′. Two adjacent stem-loops can be designed to be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 18, or at least 20 nucleotides apart from each other. Two adjacent stem-loops can be designed to he at most 200, at most 150, at most 100, at most 90, at most 80, at most 70, at most 60, at most 50, at most 40, at most 30 or at most 20 nucleotides apart from each other.

One or more terminators can be operably linked to a coding sequence such that it affects the transcription of the coding sequence. Such an operable linkage can be by way of, e.g., providing the terminator on the same DNA molecule as the coding sequence for a gene. Two or more terminators can be operatively linked if they are positioned relative to each other to provide concerted termination of a preceding coding sequence. For example, the insert can be positioned 3′ of an antisense terminator sequence and/or 5′ of a transcription terminator provided herein. In some embodiments, terminator sequences can be placed downstream of coding sequences, i.e., on the 3′ end of the coding sequence. Terminator sequences can also be upstream coding sequences. The terminator can be, e.g., at least 1, at least 10, at least 30, at least 50, at least 100, at least 150, at least 200, at least 250, at least 300, at least 400, at least 500 nucleotides downstream or upstream of the coding sequence or directly adjacent thereto. In combination thereto or independently therefrom the terminator sequence can be less than 10000, less than 8000, less than 6000, less than 5000, less than 4500, less than 4000, less than 3500, less than 3000, less than 2500, less than 2000, less than 1500, less than 1000, less than 750, less than 500, less than 250, less than 100 nucleotides downstream of the coding sequence.

In some embodiments, the present disclosure provides a non-naturally occurring nucleic acid sequence comprising a Y-X-Z stem-loop, wherein: Y is a nucleotide sequence of 10 to 30 nucleotides in length; X is a nucleotide sequence of 3 to 12 nucleotides in length, each nucleotide therein not base pairing with any other nucleotide within X; and Z is a nucleotide sequence of 10 to 50 nucleotides in length and having at least 70% complementarity to Y. X is the loop portion of the stem-loop and may be 3-8 nucleotides in length, 4-6 nucleotides in length or 5-6 nucleotides in length in some embodiments. The stem-loop in some embodiments can include the sequence of AAGC and/or CATC. In some examples, the stem-loop can have the sequence of SEQ ID NO: 3, 4, or 6.

In some embodiments, Y has a G/C content of at most 60%, at most 50%, or at most 40%. Y may be 5′ to X or 3′ to X. Y can be, in certain embodiments, 12-18 nucleotides in length, 14-16 nucleotides in length, 16-18 nucleotides in length, 17-19 nucleotides in length, 15-30 nucleotides in length, 18-27 nucleotides in length, 21-24 nucleotides in length, 24-28 nucleotides in length, or 25-29 nucleotides in length. In some embodiments, Y is of 10, 11, 12, 13, 14, 15, 16, 17, 18, 19, 20, 21, 22, 23, 24, 25, 26, 27, 28, 29, or 30 nucleotides in length.

The length of Y determines the length of Z (by complementarity), which can be selected to have substantially the same nucleotide length as Y. Z can have the same length as Y and may have one or more mismatches with Y. Z can also have one or more insertions compared to Y, thereby forming one or more protrusions or loops when annealed with Y. The length of substantially complementary Y and Z, the stem of the hairpin, determines the stem length in base pairs. The stem is not necessarily 100% complementary as described herein, but can have limited non-complementary opposing bases for Y and Z.

In particular, Y and Z can be of m and n nucleotides in length, respectively, where Y consists of nucleotides y₁, y₂ . . . to yₘ and Z consists of nucleotides z₁, z₂ . . . to zₙ. Preferably z₁ is complementary to y₁ and zₙ is complementary to yₘ so that the end points of the stem of the hairpin are complementary. Y and Z can be at least 60% complementary, preferably at least 70%, at least 80%, at least 82%, at least 84%, at least 85%, at least 86%, at least 88%, at least 90%, at least 92%, at least 94%, at least 95%, at least 96%, at least 97%, at least 98%, at least 99%, or even 100%, complementary. The complementarity is most preferably at least 70%, preferably at least 75%, at least 80%, at least 85%, at least 90%, at least 95% or 100%. Non-complementarities such as mismatches, insertions and/or deletions are possible but should be limited to meet the above complementarity percentages. Some limited non-complementarities may be placed adjacent to each other to form one or more additional loops.

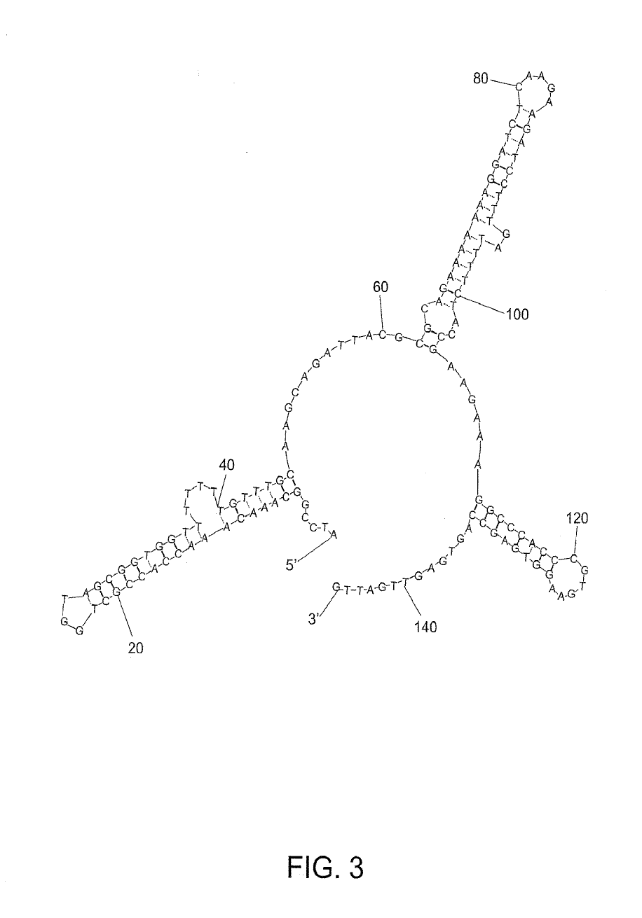

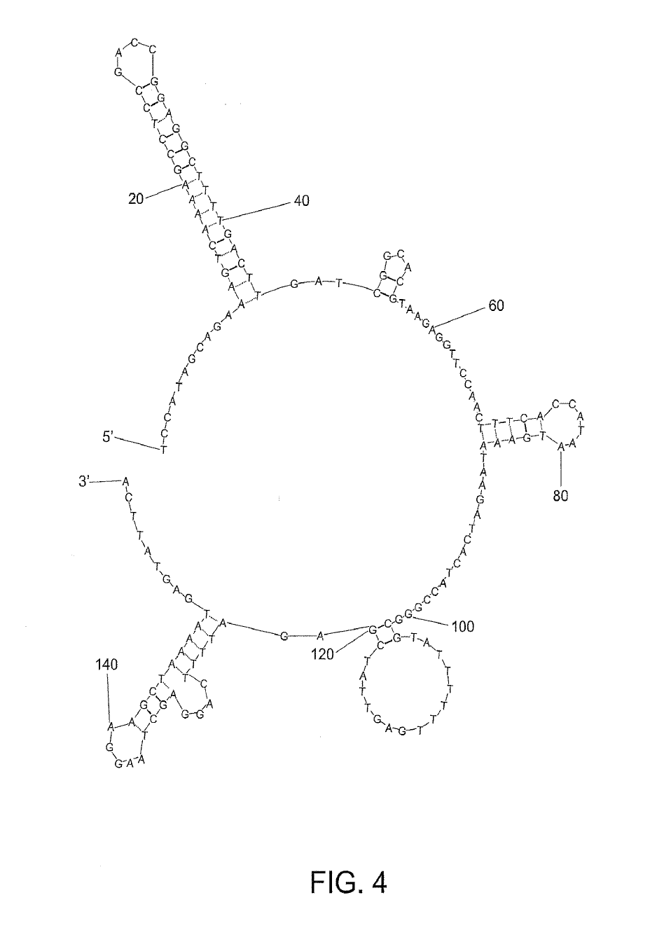

A further aspect relates to a transcription terminator comprising a first stem-loop and a second stem-loop, wherein the first stem-loop has any one of the non-naturally occurring stem-loop nucleic acid sequences disclosed herein, and wherein the first stem-loop is 5′ to the second stem-loop. In some embodiments, the second stem-loop is a short stem-loop. The second stem-loop may also have any one of the non-naturally occurring stem-loop nucleic acid sequences disclosed herein. The transcription terminator can further include a third stem-loop which can be a short stem-loop or have any one of the non-naturally occurring stem-loop nucleic acid sequence disclosed herein. An exemplary terminator of the disclosure has the sequence of SEQ ID NO: 2 or 5. Homologous terminators with at least 50%, at least 60%, at least 70%, at least 80%, at least 85%, at least 90%, at least 92%, at least 94%, at least 95%, at least 96%, at least 97%, at least 98% or at least 99% sequence identity to the terminator of SEQ ID NO: 2 or 5 are also included in the present disclosure. SEQ ID NOs: 2 and 5 describe artificial optimized terminators with several stem-loops that are underlined. Their secondary structures are shown in FIGS. 3 and 4, respectively.

### Vectors

Also provided herein is a vector comprising one or more transcription terminators disclosed herein, operably linked to a DNA insert. Where two or more terminators are included in one vector, each terminator may be placed independently from other terminators, e.g., operatively linked to an insert or a cloning site where an insert may be inserted. In some embodiments the stem-loop or the terminator is designed to be flanked by endonuclease restriction sites at its 5′ and/or 3′ terminus. Terminal restriction sites allow easy handling of the stem-loop or terminator for incorporation into other nucleic acid molecules, such as vectors or expression cassettes.

The insert can be any natural or synthetic nucleic acid sequences. In some embodiments, the insert is an in vitro synthesized or assembled nucleic acid. Various synthesis and assembly strategies are described in, for example, PCT Publication Nos. WO2014/151696, WO2014/004393, WO2013/163263, WO2013/032850, WO2012/078312, WO2004/24886, WO2008/027558, WO2010/025310, and WO2016/064856, the disclosures of all of which are hereby incorporated by reference in their entirety.

In some embodiments, following synthesis or assembly of one or more target nucleic acids, they can be individually cloned into a vector, or such cloning can be performed in a multiplex fashion in parallel.

The vector should be provided in a form suitable for easy handling, e.g., being of limited length. In some embodiments the vector comprises up to 30,000 nts (nucleotides), up to 25,000 nts, up to 20,000 nts, up to 15,000 nts, up to 12,500 nts, up to 10,000 nts, up to 9,000 nts, up to 8,000 nts, up to 7,000 nts, up to 6,000 nts.

The vector can comprise one or more genetic components such as an origin of replication, a selectable marker or antibiotic resistance gene sequence, a multiple cloning site for inserting the DNA insert, and/or a promoter, in addition to the terminator. The promoter can be operably linked with the terminator. Also included can be restriction sites flanking the terminator and/or a cloning site upstream of terminator, or an insert upstream of the terminator. Such vectors allow functionally high rates of termination during transcription of the operatively linked inserts. The terminators may be operatively positioned for termination of a transcript of a multiple cloning site (into which an insert might be inserted). The term “multiple cloning site” refers to a site comprising at least 2 sites for restriction enzymes, however, preferably it comprises a number of sites for various restriction enzymes.

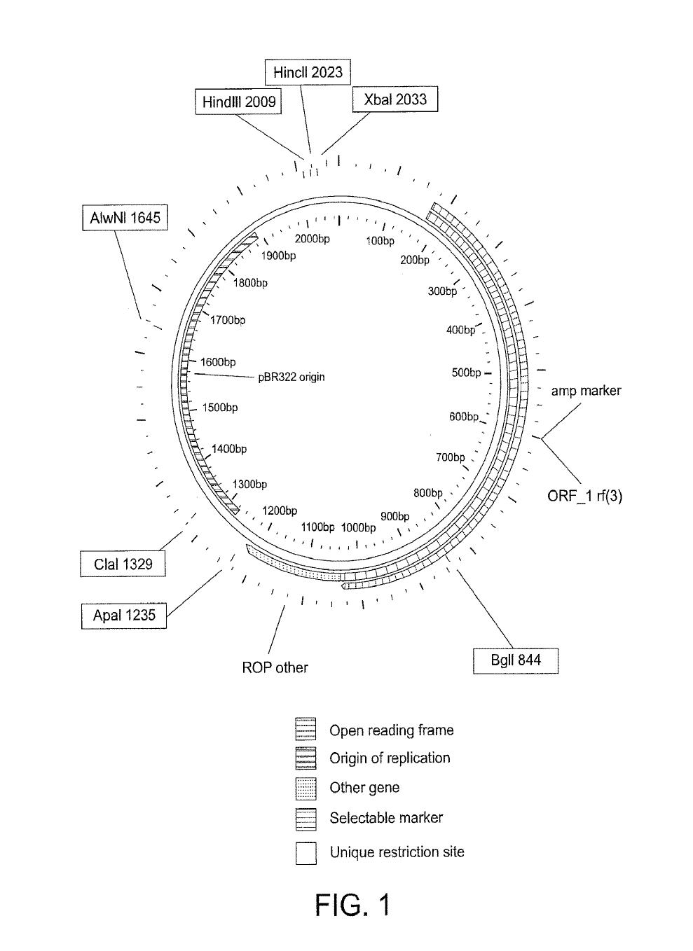

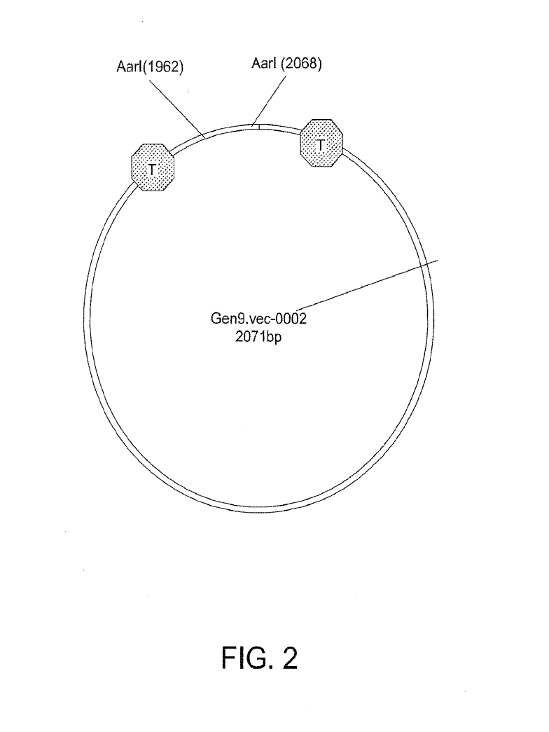

The vector in one embodiment has the sequence of SEQ ID NO: 1. FIGS. 1 and 2 are schematics of the exemplary vector.

Specifically, FIG. 1 illustrates a vector of 2071 bp in length, containing an open reading frame (ORF), a selectable marker (e.g., amp or ampicillin resistance), one or more other genes (or ORFs), a pBR322 origin and several unique restriction sites. FIG. 2 is a simplified schematic of the same vector as FIG. 1, showing the relative position of two terminators (“T”).

Another aspect relates to an engineered host cell comprising the vector disclosed herein. Host cells may be grown and expanded in culture. Host cells may be used for expressing one or more RNAs or polypeptides of interest (e.g., therapeutic, industrial, agricultural, and/or medical proteins). The expressed polypeptides may be natural polypeptides or non-natural polypeptides. The polypeptides may be isolated or purified for subsequent use. Alternatively, in vitro expression system can be used.

A further aspect related to a method of engineering a vector, comprising providing any transcription terminator disclosed herein in a vector, wherein the transcription terminator is engineered to operably link to a DNA insert.

Also provided herein is a method of terminating transcription of a DNA insert, comprising: (a) providing any transcription terminator disclosed herein engineered to operably link to the DNA insert; (b) allow transcription of the DNA insert; and (c) terminate transcription of the DNA insert at the transcription terminator.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The following examples are set forth as being representative of the present disclosure. These examples are not to be construed as limiting the scope of the disclosure as these and other equivalent embodiments will be apparent in view of the present disclosure, figures and accompanying claims.

### EXAMPLES

A low-copy, carbenicillin vector with transcription terminators is designed. The vector map is illustrated in FIGS. 1 and 2. The sequence is shown in SEQ ID NO: 1, in which the two terminators are underlined. In miniprep, about 2.5 ug of plasmids (base vector w/o insert) were collected from a 10 mL culture. The terminators have the sequences of SEQ ID NOs: 2 and 5, and their secondary structures are shown in FIGS. 3 and 4. The stem-loops are underlined in SEQ ID NOs: 2 and 5. The 3 tall stem-loops have the sequences of SEQ ID NOs: 3, 4 and 6.

SEQ ID NO: 1
actgaccatttaaatcatacctgacctccatagcagaaagtcaaaagcct
ccgaccggaggatttgacttgatcggcacgtaagaggttccaactttcac
cataatgaaataagatcactaccgggcgtattttttgagttatcgagatt
ttcaggagctaaggaagctaaaatgagtattcaacatttccgtgtcgcca
tattccatttttgcggcattttgccttcctgatttgctcacccagaaacg
ctggtgaaagtaaaagatgctgaagatcagttgggtgcacgagtgggtta
catcgaactggatctcaacagcggtaagatccttgagagtttacgccccg
aagaacgttttccaatgatgagcacttttaaagttctgctatgtggcgcg
gtattatcccgtattgacgccgggcaagagcaactcggtcgccgcataca
ctattctcagaatgacttggttgagtactcaccagtcacagaaaagcatc
tcacggatggcatgacagtaagagaattatgcagtgctgccataaccatg
agtgataacactgcggccaacttacttctggcaacgatcggaggaccgaa
ggagctaaccgcttttttgcacaacatgggggatcatgtaactcgccttg
atcgttgggaaccggagctgaatgaagccataccaaacgacgagcgtgac
accacgatgcctgtagcaatggcaacaacgttgcgcaaactattaactgg
cgaactacttactctagcttcccggcaacaattaatagactggatggagg
cggataaagttgcaggatcacttctgcgctcggccctcccggctggctgg
tttattgctgataaatctggagccggtgagcgtgggtctcgcggtatcat
tgcagcactggggccagatggtaagccctcccgcatcgtagttatctaca
cgacggggagtcaggcaactatggatgaacgaaatagacagatcgctgag
ataggtgcctcactgattaagcattggtaagtgaccaaacaggaaaaaac
cgcccttaacatggcccgctttatcagaagccagacattaacgcttctgg
agaaactcaacgagaggacgcggatgaacaggcagacatctgtgaatcgc
ttcacgaccacgctgatgagattaccgcagctgcctcgcgcgtttcggtg
atgacggtgaaaacctctgatgagggcccaaatgtaatcacctggctcac
cttcgggtgggcctttctgcgttgctggcgtttttccataggctccgccc
ccctgacgagcatcacaaaaatcgatgctcaagtcagaggtggcgaaacc
cgacaggactataaagataccaggcgtttccccctggaagctccctcgtg
cgctctcctgttccgaccctgccgcttaccggatacctgtccgcattctc
catcgggaagcgtggcgctttacatagctcacgctgtaggtatctcagtt
cggtgtaggtcgttcgctccaagctgggctgtgtgcacgaaccccccgtt
cagcccgaccgctgcgccttatccggtaactatcgtcttgagtccaaccc
ggtaagacacgacttatcgccactggcagcagccactggtaacaggatta
gcagagcgaggtatgtaggcggtgctacagagttcttgaagtggtggcct
aactacggctacactagaagaacagtatttggtatctgcgctctgctgaa
gccagttacctcggaaaaagagttggtagctcttgatccggcaaacaaac
caccgctggtagcggtggtttttttgtttgcaagcagcagattacgcgca
gaaaaaaaggatctcaagaagatcctttgattttctaccgaagaaaggcc
cacccgtgaaggtgagccagtgagttgattgcagtccagttacgctggag
tcaagcagctgcaggtgtgtgtgtgtgaggctcgtcctgaatgatatcaa
gcttgaattcgttgacgaattctctagatatcgctcaatcacacacacac
ctgcagctcatc
(5′-Terminator)
SEQ ID NO: 2
ATCCGGCAAACAAACCACCGCTGGTAGCGGTGGTTTTTTTGTTTGCAAGC
AGCAGATTACGCGCAGAAAAAAAGGATCTCAAGAAGATCCTTTGATTTTC
TACCGAAGAAAGGCCCACCCGTGAAGGTGAGCCAGTGAGTTGATTG
SEQ ID NO: 3
GCAAACAAACCACCGCTGGTAGCGGTGGTTTTTTTGTTTGC
SEQ ID NO: 4
CGCAGAAAAAAAGGATCTCAAGAAGATCCTTTGATTTTCTACCG
(3′-Terminator)
SEQ ID NO: 5
TCCATAGCAGAAAGTCAAAAGCCTCCGACCGGAGGCTTTTGACTTGATCG
GCACGTAAGAGGTTCCAACTTTCACCATAATGAAATAAGATCACTACCGG
GCGTATTTTTTGAGTTATCGAGATTTTCAGGAGCTAAGGAAGCTAAAATG
AGTATTCA
SEQ ID NO: 6
AAGTCAAAAGCCTCCGACCGGAGGCTTTTGACTT

### Equivalents

The present disclosure provides among other things novel methods and systems for improved cloning efficiency using synthetic transcription terminator(s). While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### Incorporation by Reference

The ASCII text file submitted herewith via EFS-Web, entitled “127662015201SequenceListing.txt” created on Nov. 29, 2016, having a size of 4,285 bytes, is incorporated herein by reference in its entirety.

All publications, patents and sequence database entries mentioned herein are hereby incorporated by reference in their entirety as if each individual publication or patent was specifically and individually indicated to be incorporated by reference.

1. A non-naturally occurring nucleic acid sequence comprising a Y-X-Z stem-loop, wherein:
Y is a nucleotide sequence of 10 to 30 nucleotides in length;
X is a nucleotide sequence of 3 to 12 nucleotides in length, each nucleotide therein not base pairing with any other nucleotide within X; and
Z is a nucleotide sequence of 10 to 50 nucleotides in length and having at least 70% complementarity to Y.

2. The non-naturally occurring nucleic acid sequence of claim 1, wherein Y has a G/C content of at most 60%, preferably at most 50%, more preferably at most 40%.

3. The non-naturally occurring nucleic acid sequence of claim 1, wherein Y is engineered to be 5′ to X.

4. The non-naturally occurring nucleic acid sequence of claim 1, wherein Y is engineered to be 3′ to X.

5. The non-naturally occurring nucleic acid sequence of claim 1, wherein Y is 12-18 nucleotides in length, or 14-16 nucleotides in length, or 16-18 nucleotides in length, or 17-19 nucleotides in length, or 15-30 nucleotides in length, or 18-27 nucleotides in length, or 21-24 nucleotides in length, or 24-28 nucleotides in length, or 25-29 nucleotides in length.

6. The non-naturally occurring nucleic acid sequence of claim 1, wherein X is 3-8 nucleotides in length, preferably 4-6 nucleotides in length, more preferably 5-6 nucleotides in length.

7. The non-naturally occurring nucleic acid sequence of claim 1, wherein Z has the same length as Y and has one or more mismatches with Y or wherein Z has a different length than Y and has one or more insertions or deletions compared to Y.

8. (canceled)

9. The non-naturally occurring nucleic acid sequence of claim 1, comprising AAGC or comprising CATC.

10. (canceled)

11. The non-naturally occurring nucleic acid sequence of claim 1, having the sequence of SEQ ID NO: 3, 4, or 6.

12. A transcription terminator comprising a first stem-loop and a second stem-loop, wherein the first stem-loop has the non-naturally occurring nucleic acid sequence of claim 1, and wherein the first stem-loop is engineered to be 5′ to the second stem-loop.

13. The transcription terminator of claim 12, wherein the second stem-loop is a short stem-loop.

14. The transcription terminator of claim 12, wherein the second stem-loop has the non-naturally occurring nucleic acid sequence of claim 1.

15. The transcription terminator of claim 12, further comprising a third stem-loop, optionally wherein the third stem-loop is a short stem-loop.

16. (canceled)

17. The transcription terminator of claim 15, wherein the third stem-loop has the non-naturally occurring nucleic acid sequence of claim 1.

18. The transcription terminator of claim 12, having the sequence of SEQ ID NO: 2 or 5.

19. A vector comprising the transcription terminator of claim 12, wherein the transcription terminator is operably linked to a DNA insert.

20. The vector of claim 19, having the sequence of SEQ ID NO: 1.

21. An engineered cell comprising the vector of claim 19.

22. A method of engineering a vector, comprising providing the transcription terminator of claim 12 in a vector, wherein the transcription terminator is engineered to operably link to a DNA insert.

23. A method of terminating transcription of a DNA insert, comprising:
a. providing the transcription terminator of claim 12 engineered to operably link to the DNA insert;
b. allow transcription of the DNA insert; and
c. terminate transcription of the DNA insert at the transcription terminator.
